(19) United States
(12) Patent Application Publication
(10) Pub. No.: US 2019/0133917 A1
(43) Pub. Date: May 9, 2019

(54) FRAGRANCE PRIMER COMPOSITIONS AND METHODS

(71) Applicant: Scentinvent Technologies LLC, Scarsdale, NY (US)

(72) Inventors: Abby Wallach, Scarsdale, NY (US); Caroline Pieper-Vogt, Scarsdale, NY (US); Steven Semoff, New City, NY (US)

(21) Appl. No.: 16/088,629

(22) PCT Filed: Apr. 28, 2017
(86) PCT No.: PCT/US17/30168

§ 371 Date: Sep. 26, 2018

Related U.S. Application Data
(60) Provisional application No. 62/329,027, filed on Apr. 28, 2016.

(51) Int. Cl. A61K 8/891; A61Q 13/00; A61K 8/34; A61K 8/55; A61K 8/81

(52) U.S. Cl. CPC A61K 8/891; A61Q 13/00; A61K 8/8152; A61K 8/553; A61K 8/55; A61K 8/345

(57) ABSTRACT

The present technology relates to compositions useful for “priming” the surface of a user's skin before, contemporaneous with, or after application of a compound containing a fragrance to the skin, to prolong the longevity of the fragrance. The present technology is also directed to methods of reducing the dissipation, or the rate of dissipation, of a fragrance from the surface of the skin.

### BACKGROUND

The present technology relates to compositions useful for application to the skin in conjunction with fragrant compounds such as perfumes, in order to prolong the efficacy of such fragrant compounds. In particular, the present technology relates to compositions and methods for priming the skin in order to prolong the efficacy of a fragrance after application beyond that of unprimed skin.

Fragrances such as perfumes, colognes, eau de toilette and the like are often applied to the skin as part of skin care or cosmetic routines, either as a fragrance alone or in the form of other skin care compositions, such as lotions, powders, cosmetics or sunscreens that contain a fragrance.

However, one common problem is that the desired fragrance often does not last—that is, the fragrance often dissipates within a short period of time, necessitating frequent reapplication of the fragrant compound. Over time, this is inconvenient and can lead to higher costs for the consumer as the product is used up more rapidly over time. Fragrances such as cologne are based on ethanol (ethyl alcohol) at a level of approximately 75%. Ethanol is exceptionally drying to skin and may defat the skin leaving white dry marks on the surface of the skin. Thus, a need exists for improved compositions and methods that permit a consumer to apply a fragrance in a manner that slows down the overall dissipation (which can be the overall dissipation or the rate of dissipation over a certain period of time) of the fragrance from the skin, thus prolonging the consumer's enjoyment of the fragrance, and avoiding drying the skin and the need for frequent reapplication.

### SUMMARY OF THE DISCLOSED TECHNOLOGY

In certain embodiments, the present technology is directed to a composition (also referred to herein as a “primer composition”) that is applied to the skin separately from a fragrance, the composition comprising: (a) a barrier agent; (b) a hydroscopic agent (also known as a humectant); and (c) a rheology agent. The composition is applied to the skin before application of the fragrance and can reduce the dissipation of the fragrance from the skin.

In certain embodiments, the present technology is directed to methods of reducing the dissipation of a fragrance from the surface of the skin, or of prolonging the presence of a fragrance on the skin of a user, the methods comprising applying the compositions herein to the skin either before or contemporaneous with applying the fragrance to the skin.

### DETAILED DESCRIPTION

As used herein, “fragrant compound” or “fragrance” are used interchangeably and can refer to either a fragrance alone or with solvents, such as a perfume, eau de cologne or eau de toilette, as well as a fragrance incorporated within additional ingredients in the form of a cosmetic such as a lotion, powder, liquid, suspension, gel, spray, vapor or atomized droplet. As used herein, “skin” means skin, hair, scalp, nails or any external surface of a patient's body. As used herein, “applied to” means contacted with in any amount. As used herein, “contemporaneous with” means at exactly or approximately the same time—that is, when discussing embodiments wherein the primer compositions herein are applied contemporaneous with applying a fragrance or fragrant compound to the skin, this encompasses embodiments wherein the primer composition and the fragrance or fragrant compound are applied together as part of a single application or a single formulation; or whether one or the other (either the primer composition or the fragrant compound) is applied within a short period of time before the other—including, e.g., within up to 5 seconds, up to 10 seconds, up to 30 seconds, up to 45 seconds or up to 1 minute beforehand. All percentages used herein are by weight unless otherwise noted.

Fragrance is generally built according to the following pattern: top note, also called a head note; a middle note, also called a heart note; and a back note, also called a base note. Generally, the top note is the first to be detected by the nose, followed by the middle note and finally the base note as time passes. Often, when a fragrance is applied to the surface of the skin, openings in the surface of the skin will cause some of the notes to be absorbed into the skin rather than dissipated to the environment. This generally decreases the amount of fragrance that is available for dissipation into the environment and the consumer's enjoyment of the fragrance.

Compositions have been developed herein that “prime” the skin in a manner similar to that of a base coat of paint on a wall—that is, the compositions can cover the surface of the skin, thereby minimizing the amount of fragrance that is absorbed into the skin (and thus lost), and optimizing the amount of fragrance that remains on the surface and open to the surrounding environment, which fragrance can thus continue emitting into the environment for the olfactory enjoyment of the consumer.

Application of Compositions

In certain embodiments, the compositions herein are in any form that will permit easy application to the skin before or contemporaneous with application of the fragrant compound. For example, in various embodiments, the compositions herein can be in the form of a spray, mist, vapor, atomized droplet, lotion, cream, salve, paste, suspension, gel, emulsion, powder, foam, solid stick or any other cosmetically suitable form that is readily applied by a consumer. In certain embodiments, the compositions herein are in entirely liquid form, entirely solid form, or in any combination of gas, liquid and solid form. In certain embodiments, the compositions can be in the form of a spray, mist or atomized droplet, e.g., in a spray bottle, and applied in the same manner as a perfume, cologne or fragrance is typically applied. A user can keep a bottle of the primer composition near his or her fragrance, and spray both the primer composition and the fragrance onto his or her body as part of the same beauty or grooming routine. In other embodiments, the primer composition need not be in the same form as a fragrance, but could be, for example, a solid stick, paste or lotion that could be applied either with a hand or an applicator.

In certain embodiments, the compositions can be applied directly onto the skin either: (1) before; (2) contemporaneous with; or (3) after the application of the fragrant compound. In certain embodiments, the compositions can be applied to the skin, and allowed to penetrate into the skin to smooth and cover the surface of the skin, in a manner similar to that of a base coat of paint. When the fragrant compound is thereafter applied to the composition, the surface of the skin is optimally smooth and free of imperfections that might otherwise interfere with the release of the top notes, middle notes or base notes. In certain embodiments, the compositions can be applied to the skin after the fragrant compound is applied to the skin; in certain embodiments, after more than 1 minute, after more than 2 minutes, after more than 5 minutes or after more than 10 minutes.

Barrier Agent

In certain embodiments, the compositions herein comprise a barrier agent, also known as an aesthetic modifier. An exemplary barrier agent herein contains one or more of the following ingredients: dimethicone, liquid paraffin, lanolin, polydimethyl siloxane, a fatty acid, propylene glycol, butylene glycol and hydrogenated lecithin. The barrier agent can impart desirable properties to the compositions including acting as a film former, retaining moisture on the skin, reducing trans-epidermal water loss through the skin, reducing irritation of the skin, increasing stability and otherwise exhibiting ease of application to the consumer's skin. In certain embodiments, the barrier agent can be partially or completely waterproof or water resistant.

In various embodiments, one or more barrier agents are present in the compositions in amounts of about 2 to about 20%, about 5 to about 15%, about 8 to about 12%, about 9 to about 11% or about 10%.

Rheology Agent

In certain embodiments, the compositions herein further comprise a rheology agent, also known as an emulsifier or thickening agent. The agent can help to maintain the compositions in a desired state—for example, in certain embodiments, the compositions have a viscosity at room temperature (about 20° C. to about 30° C., or about 25° C.) of about 5 to about 300 centistokes, about 10 to about 200 centistokes, or about 15 to about 175 centistokes, to maintain ease of application to a consumer's skin, either by, e.g., spraying, misting, rubbing, slathering, painting with a brush, dabbing, applying with a roller or otherwise applying to the skin in a manner that will cover the skin and provide the desirable “priming” characteristics to optimize the staying power of the fragrant compound.

In certain embodiments, the rheology agent is a combination of two or more compositions—for example, a combination of acrylates/C10-30 alkyl acrylate crosspolymer and potassium cetyl phosphate. In other embodiments, the rheology agent is a single composition that can provide both emulsifying and thickening properties, for example, a solution of hydroxyethyl acrylate, sodium acryloyldimethyl taurate copolymer and a non-ionic surface active agent or polyacrylamide (and) C13-14 isoparaffin (and) Laureth-7 as a blend in water.

In various embodiments, one or more emulsifiers or thickeners are present in the compositions in amounts of about 0.1 to about 2%, about 0.25 to about 0.8%, about 0.4 to about 0.75%, about 0.5%, about 0.6% or about 0.75%.

Hydroscopic Agent/Humectant

In certain embodiments, the compositions herein further comprise a hydroscopic agent, also known as a humectant. Suitable examples include polyhydric alcohols such as propanediol, propylene glycol, hexylene glycol, butylene glycol, glyceryl triacetate; sugar alcohols such as glycerol, sorbitol, xylitol or maltitol; and polymeric polyols such as polyethylene glycol (PEG). In various embodiments, one or more humectants or hydroscopic agents are present in the present compositions in amounts of about 1 to about 5%, about 1.25 to about 4%, about 1.5 to about 3% or about 1.75 to about 2.5%.

Additional Ingredients

In certain embodiments, the compositions herein further comprise a neutralizing agent, to bring the compositions to an optimal pH. In various embodiments, the optimal pH of the compositions is about 3 to about 10, about 4 to about 9, about 5 to about 8 or about 5.5 to about 7.5. Useful neutralizing agents include, for example, sodium hydroxide, ammonium hydroxide, potassium hydroxide, arginine, aminomethyl propanol, tetrahydroxypropyl ethylenediamine, triethanolamine, triisopropanolamine or sodium citrate. In various embodiments, one or more of these additional ingredients can be present in amounts of about 0.01 to about 0.75%, about 0.05 to about 0.5 or about 0.1 to about 0.25%.

In certain embodiments, the compositions herein further comprise a preservative. Exemplary preservatives include those containing any of the following: aromatic ether alcohols or acids. For example, useful preservatives include various combinations of phenoxyethanol, caprylyl glycol and acids; as well as those containing propylene glycol, propylparaben, methylparaben or diazolidinyl urea. In various embodiments, one or more preservatives are present in the compositions in amounts of about 0.1 to about 1.5%, about 0.25 to about 1% or about 0.4 to about 0.75%.

In various embodiments, the primer compositions herein can themselves include a fragrance, either the same as, or different from the fragrant compound that the user desires to be prolonged on his or her skin. In other embodiments, the primer compositions can be fragrance free—that is, exhibit no discernible fragrance, or no fragrance that would be in amounts significant enough to interfere with the scent of the user's desired fragrant compound.

The following non-limiting Examples illustrate certain embodiments contemplated by the various embodiments herein.

### Example 1

A composition was prepared as set forth in Table 1:

TABLE 1

| Ingredient | Weight percentage (%) |
|---|---|
| Deionized water | about 75 to about 90 |
| Water (and) Dimethicone (and) Dimethicone | about 5 to about 15 |
| Crosspolymer (and) Butylene Glycol (and) | |
| Hydrogenated Lecithin (and) | |
| Polyphosphorylcholine Glycol Acrylate | |
| Phenoxyethanol (and) Caprylyl Glycol (and) | about 0.1 to about 2 |
| Sorbic Acid | |
| Propanediol | about 1 to about 5 |
| Emulsifier/thickener | about 0.25 to about 0.75 |

### Example 2

A composition was prepared as set forth in Table 2:

TABLE 2

| Ingredient | Weight percentage (%) |
|---|---|
| Deionized water | about 80 to about 88 |
| Water (and) Dimethicone (and) Dimethicone | about 5 to about 15 |
| Crosspolymer (and) Butylene Glycol (and) | |
| Hydrogenated Lecithin (and) | |
| Polyphosphorylcholine Glycol Acrylate | |
| Phenoxyethanol (and) Caprylyl Glycol (and) | about 0.1 to about 2 |
| Sorbic Acid | |
| Propanediol | about 1 to about 5 |
| Emulsifier/thickener | about 0.5 to about 1 |

### Example 3

A composition was prepared as set forth in Table 3:

TABLE 3

| Ingredient | Weight percentage (%) | Phase |
|---|---|---|
| Deionized water | about 40 to about 50 | A |
| Acrylates/C10-30 Alkyl Acrylate Crosspolymer | about 0.01 to about 1 | |
| Deionized water | about 35 to about 45 | B |
| Potassium Cetyl Phosphate | about 0.1 to about 1 | |
| Water (and) Dimethicone (and) Dimethicone | about 5 to about 15 | C |
| Crosspolymer (and) Butylene Glycol (and) Hydrogenated | | |
| Lecithin (and) Polyphosphorylcholine Glycol Acrylate | | |
| Phenoxyethanol (and) Caprylyl Glycol (and) Sorbic Acid | about 0.1 to about 2 | D |
| Propanediol | about 1 to about 5 | |
| Triethanolamine 99% | about 0.05 to about 0.5 | E |

The ingredients of Phase A were blended at high shear at 25° C. until a uniform solution was obtained. Phase B was prepared by heating the water to a temperature of above 75° C. and adding the potassium cetyl phosphate until a solution was obtained. Phases A and B were blended with propeller mixing until uniform. Phases C, D and E were added under further propeller mixing until a uniform liquid emulsion was obtained.

### Example 4

A composition was prepared as set forth in Table 4:

TABLE 4

| Ingredient | Weight percentage (%) |
|---|---|
| Deionized water | about 80 to about 90 |
| Water (and) Dimethicone (and) Dimethicone | about 5 to about 15 |
| Crosspolymer (and) Butylene Glycol (and) | |
| Hydrogenated Lecithin (and) | |
| Polyphosphorylcholine Glycol Acrylate | |
| Propanediol | about 1 to about 5 |
| Rheology Agent | about 0.5 to about 1 |

A comparison was made between the formula of Example 3 and the formula of

Example 4, both of which are contemplated by the present disclosure.

Both formulas include water; a barrier agent that includes a silicone nano-emulsion; a preservative that prevents bacterial growth in the package; a hydroscopic agent also known as a humectant; as well as one or more rheology agents that are included to keep the formula homogenous and prevent creaming, separation and precipitation during storage.

In certain embodiments, the formula of Example 3 can be made through heating a portion of the water to above 75° C., above 80° C. or above 85° C. in order to dissolve the one or more rheology agents. One or more of the rheology agents can be an acid based acrylic thickener that is neutralized with a neutralizing agent such as triethanolamine. The neutralization can result in thickening of the formula to prevent separation upon storage. In contrast, the formula of Example 4 can be made at room temperature (about 20° C. to about 30° C., or about 25° C.) in a single kettle under propeller mixing, in certain embodiments by using a cold process rheology agent comprising a blend of hydroxyethyl acrylate, sodium acryloyldimethyl taurate, isohexadecane and polysorbate 60.

### Example 5

A study was performed to determine the potential of a primer composition (test product) in accordance with the embodiments herein, to extend the scent of fragrances. The study was conducted in accordance with the intent and purpose of Good Clinical Practice regulations of Title 21 of the U.S. Code of Federal Regulations (21 C.F.R.), and the Declaration of Helsinki and/or Essex Testing Clinic (ETC) Standard Operation Procedures. The study was conducted in compliance with 21 C.F.R. § 50 (Informed Consent of Human Subjects).

The test panel was made up of 30 female human subjects, aged 18 to 70 years. The subjects were in general good health; free of any skin conditions or history of skin conditions that might interfere with the procedure; not taking any steroidal/non-steroidal anti-inflammatory drugs or antihistamines; and having no known sensitivity to cosmetics or personal care products.

For each subject, a single application of the primer composition (white viscous liquid) was applied to one wrist as a spray mist, and a fragrance was then applied to the same wrist. The fragrance was also applied to the subject's other wrist (absent the primer composition). Just after application, the trained evaluator evaluated the scent and strength of each fragrance on each wrist.

Each subject was evaluated again by the trained evaluator at 1, 2, 4, 6 and 8 hours following application.

Evaluations were conducted according to the following scale:

5=strong scent

4=mild scent

3=moderate scent

2=light scent

1=no scent

Evaluation of efficacy was based on a comparison of baseline versus each observation period. A comparison was made between the sites treated with fragrance+primer composition, versus fragrance alone.

After 8 hours, the subjects completed a questionnaire about their experiences with the primer composition. 93% of the subjects reported the primer composition as feeling desirably “smooth and dry” and not rough (rather than undesirably “wet and sticky” or “oily and shiny”) when contacting the skin.

70% of the subjects reported that the primer composition helped the fragrance to wear longer. 63% of the subjects reported a prolonged fragrance of 3 to 5 hours; 20% reported a prolonged fragrance of 3 hours, and 17% reported a prolonged fragrance of 2 hours.

When asked how many times the primer composition “extend[ed] your fragrance wear,” 27% said 2 times longer, 43% said 3 times longer, and 30% said 3 to 5 times longer.

When asked whether they agreed that the primer composition “helped your fragrance to say true,” 37% strongly agreed and 50% moderately agreed.

When asked whether they agreed that the primer composition spray mist function “make[s] for easy and fast application,” 77% said Yes.

The evaluator's data showed that when the primer composition+fragrance was compared with the fragrance alone, the primer composition+fragrance extended the scent of the fragrance at 1, 2, 4, 6 and 8 hours after application. Table 5 shows the mean score of the fragrance alone versus the mean score of the primer composition+fragrance.

TABLE 5

| | | | Primer Composition + | | |
|---|---|---|---|---|---|
| | Fragrance Alone | | Fragrance | | |
| | Baseline | | | | |
| | | Mean % | | Mean % | % |
| | 4.8 | Change | 4.7 | Change | Improvement |
| | Mean | from | Mean | from | by Using |
| | Score | Baseline | Score | Baseline | Primer |
| 1 hour | 4.4 | 8% | 4.5 | 4% | 2% |
| 2 hours | 3.6 | 25% | 3.7 | 21% | 3% |
| 4 hours | 2.9 | 40% | 3.0 | 36% | 3% |
| 6 hours | 2.2 | 54% | 2.5 | 47% | 14% |
| 8 hours | 1.3 | 73% | 1.8 | 62% | 38% |

The last column on the far right shows the calculated percentage of improvement of the mean score achieved by using the primer. For example, at one hour, the mean score for the fragrance alone was 4.4, while the mean score for the primer composition+fragrance was 4.5. The difference between the two (0.1) was divided by the mean score for the fragrance alone to arrive at a percentage of improvement by using the primer: that is, 0.1/4.4=2%. This was done for each time interval (1 hour, 2 hours, 4 hours, 6 hours, 8 hours). Thus, the far right column shows that by using the primer composition, the improved retention/prolonging (or put another way, the decreased dissipation) of the scent was at least 2% after 1 hour, at least 3% after 2 hours, at least 3% after 4 hours, at least 14% after 6 hours, and at least 38% at 8 hours, versus using the fragrance alone.

Table 6 shows a side-by-side comparison of the percentage of decrease of scent for the fragrance alone versus the primer composition+fragrance:

TABLE 6

| | Percentage Decrease of Scent | | | | |
|---|---|---|---|---|---|
| | 1 hour | 2 hours | 4 hours | 6 hours | 8 hours |
| Fragrance alone | 8% | 25% | 40% | 54% | 73% |
| Primer composition + | 4% | 21% | 36% | 47% | 62% |
| Fragrance | | | | | |

As can be seen here, when subjects used the fragrance alone, they reported, on average, a 73% reduction in the amount of fragrance 8 hours after application. However, when subjects used the fragrance+primer composition, that reduction at 8 hours after application was only, on average, 62%.

Thus, it can be seen that using the primer composition contemporaneous with the fragrance led to a noticeably longer retention/prolonging of the fragrance on the subject's skin.

Other advantages of the compositions herein include low cost and ease of manufacture, easy portability, and stability of formulation.

Although the present technology has been described in relation to particular embodiments thereof, these embodiments and examples are merely exemplary and not intended to be limiting. Many other variations and modifications and other uses will become apparent to those skilled in the art. The present technology should, therefore, not be limited by the specific disclosure herein, and may be embodied in other forms not explicitly described here, without departing from the spirit thereof.

1. A primer composition comprising:
(a) a barrier agent;
(b) a hydroscopic agent; and
(c) a rheology agent;
wherein the primer composition, when applied to the skin of a user before, contemporaneous with or after the application of a fragrance to the skin of the user, slows down the dissipation of the fragrance from the skin of the user.

2. The primer composition of claim 1, wherein the barrier agent comprises dimethicone, liquid paraffin, lanolin, polydimethyl siloxane, a fatty acid, propylene glycol, butylene glycol or hydrogenated lecithin.

3. The primer composition of claim 1, wherein the barrier agent is present in an amount of about 2 to about 20% of the primer composition.

4. The primer composition of claim 1, wherein the hydroscopic agent comprises a polyhydric alcohol, a sugar alcohol or a polymeric polyol.

5. The primer composition of claim 1, wherein the hydroscopic agent is present in an amount of about 1 to about 5% of the primer composition.

6. The primer composition of claim 1, wherein the rheology agent comprises a combination of acrylates/C10-30 alkyl acrylate crosspolymer and potassium cetyl phosphate.

7. The primer composition of claim 1, wherein the rheology agent is present in an amount of about 0.1 to about 2% of the primer composition.

8. The primer composition of claim 1, wherein the rheology agent comprises a solution of hydroxyethyl acrylate, sodium acryloyldimethyl taurate copolymer and a non-ionic surface active agent or polyacrylamide (and) C13-14 isoparaffin (and) Laureth-7 as a blend in water.

9. The primer composition of claim 1, further comprising one or more of a neutralizing agent or a preservative.

10. The primer composition of claim 1, in the form of a spray, mist, vapor, atomized droplet, lotion, cream, salve, paste, suspension, gel, emulsion, powder, foam or solid stick.

11. A composition comprising a primer composition of claim 1, mixed with a fragrant compound.

12. A method of reducing the dissipation of a fragrance from the skin of a user, the method comprising applying a primer composition of claim 1 to the skin of a user either before or contemporaneous with applying a fragrance to the skin of the user.

13. A method of prolonging the presence of a fragrance on the skin of a user, the method comprising applying a primer composition of claim 1 to the skin of a user either before or contemporaneous with applying a fragrance to the skin of the user.

14. The method of claim 13, wherein at 6 hours after application, the dissipation of the fragrance from the skin of the user is reduced by at least about 14% when compared with the dissipation of the fragrance when applied alone to the skin of the user without the primer composition.

15. The method of claim 13, wherein at 8 hours after application, the dissipation of the fragrance from the skin of the user is reduced by at least about 38% when compared with the dissipation of the fragrance when applied alone to the skin of the user without the primer composition.
